(19) United States
(12) Patent Application Publication
(10) Pub. No.: US 2021/0187858 A1
(43) Pub. Date: Jun. 24, 2021

(54) METHOD AND SYSTEM FOR MANUFACTURING OF PHARMACEUTICAL FORMULAS IN FORM OF ORALLY DISINTEGRATING FILMS (ODF)

(71) Applicant: DIEU CAM VUONG, (US)

(72) Inventor: DIEU CAM VUONG, MILPITAS, CA (US)

(73) Assignee: DIEU CAM VUONG, MILPITAS, CA (US)

(21) Appl. No.: 16/719,996

(22) Filed: Dec. 19, 2019

Related U.S. Application Data
(63) Continuation-in-part of application No. 16/719,901, filed on Dec. 18, 2019.

(51) Int. Cl. B29C 64/393; A61K 9/00; B33Y 30/00; B33Y 80/00; B33Y 50/02

(52) U.S. Cl. CPC B29C 64/393; A61K 9/0056; B33Y 70/00; B33Y 80/00; B33Y 50/02; B33Y 30/00

(57) ABSTRACT

A system and method of manufacturing a formula in form of orally disintegrating films (ODF) is disclosed which comprises means for preparing the formula; a printer for generating the formula in form of a matrix ODF, wherein the matrix ODF further comprises a plurality of ODF units each containing such formula and having xyz-coordinates; and a scanner for detecting defects in the plurality of ODF units.

### CLAIM OF PRIORITY

This application is a continuation application-in-part (CIP) under 35 U.S.C. § 120 of application Ser. No. 16/719,901, entitled “Heat And Oxidation Resistant Δ9 Tetrahydrocannobinol (THC) And Cannabinol (CBN) Compound And Method Of Manufacturing the Same”, filed on Dec. 18, 2019, application patent Ser. No. 16/719,901. The patent application identified above is incorporated here by reference in its entirety to provide continuity of disclosure.

### FIELD OF THE INVENTION

The present invention relates generally to an orally disintegrating film (ODF). More specifically, the present invention relates to a self-correcting method and system for manufacturing ODF formulas.

### BACKGROUND ART

Recent research has found that when a drug is admitted sub-lingually (under the tongue), it will absorb into the blood stream more efficiently than the traditional swallowing. This is because the stomach acids destroy a decent amount of the admitted drugs. Thus, there are different methods of manufacturing orally disintegrating films, or orally dissolving films (both abbreviated to ODF) for drugs and foods in the markets. However, these conventional methods and systems involves complex machinery, expensive, and are low in efficiency.

Referring now to FIG. 1 which illustrates a prior art casting production system and method 100 of cannabis drugs. Casting production system and method 100 involves a casting step 101, a drying step 102, a vision inspection step 103, a sealing step 104, a defect punching step 105, a printing/slitting step 106, a cutting step 107, and a packaging step 108. As seen, the prior-art casting production system and method 100 involves 8 different steps.

At step 101, a 12-nozzles casting machine is used to cast or paste the heat and oxidation resistant Tetrahydrocannabinol (THC) and Cannabidiol Composition (hereinafter referred to as “cannabinoid formula e”) in the form of an ODF on a substrate.

At step 102, the cannabinoid formula ODF is undergoes drying.

At step 103, each cannabinoid formula ODF is inspected for defects and/or air bubbles. Air bubbles are formed during the process of casting (pasting) at the nozzles.

At step 104, the cannabinoid formula ODF is sealed off to prevent further air bubbles from forming.

At step 105, the defective cannabinoid formula ODF is punched marked where the defects are found.

At step 106, the cannabinoid formula ODF is printing/splitting to include the name, ingredients, and usage of the cannabinoid formula ODF.

At step 107, the ODF cannabis is cut, and defective ones are eliminated.

Finally, at step 108, the cannabinoid formula ODF is packaged and shipped.

Prior art casting production system and method 100 involves 12 nozzle casting, tedious visual inspection of each ODF package, and punching to mark where the defects occur. At step 107, the defective ODF are discarded without fixing them. Thus, prior art casting production system and method 100 has efficiency below 80% and involves expensive systems and labor to manufacture ODF products.

Furthermore, prior art casting production system and method 100 cannot produce different geometrical shapes for each cannabinoid formula ODF because casting production can only produce rectangular shaped ODFs.

Furthermore, prior art casting production system and method 100 cannot control the thickness and dimension of each cannabinoid formula ODF. Higher dosages of cannabinoid formula ODF will require more cannabis mixture raw material in each ODF, which may cause the casting machine to smear while casting the ODFs and increases the likelihood of defects such as air bubbles being present on the ODF units.

Finally, prior art casting production system and method 100 cannot produce cannabinoid formula ODF and track the location of each cannabinoid formula ODF. The defects in the cannabinoid formula ODF have to be punch marked as a form of tracking defective ODF units.

Therefore what is needed is method and system that involves a fewer steps and more cost-effective than the prior art casting production to manufacture ODF products.

What is needed is a method and system for manufacturing ODF products that can correct defects without discarding them, thus improving efficiency.

What is needed is a method and system for manufacturing ODF products that can control the numbers, the dimension, and the geographical shapes of the ODF products.

What is needed is a method and system for manufacturing ODF products that can use advanced printing technology such as 4D printing.

### SUMMARY OF THE INVENTION

Accordingly, an object of the present invention is to provide a system of manufacturing a formula in the form of ODF which comprises the means for preparing the formula; a printer for generating the formula in the form of a matrix ODF, wherein the matrix ODF further comprises a plurality of ODF units each containing such formula and having xyz-coordinates (pursuant to the Cartesian coordinate system); and a scanner for detecting defects in the plurality of ODF units.

Another object of the present invention is to provide a method manufacturing a formula in form of ODF that includes: preparing the formula; coordinated matrix forming the formula in form of a plurality of ODF units, each having a geometrical shape and xyz-coordinates; scanning for defects in the plurality of ODF units; and reforming the plurality of ODF units that are defected at the exact xyz-coordinates of defects.

Another object of the present invention is to provide a simple and defect-free or close to defect-free method and system for manufacturing cannabinoid formula ODFs.

Another object of the present invention is to provide a self-correcting method and system of manufacturing ODF formulas.

### BRIEF DESCRIPTION OF THE DRAWINGS

The accompanying drawings, which are incorporated in and form a part of this specification, illustrate embodiments of the invention and, together with the description, serve to explain the principles of the invention.

FIG. 1 is a flow chart illustrating a prior-art casting production of ODF;

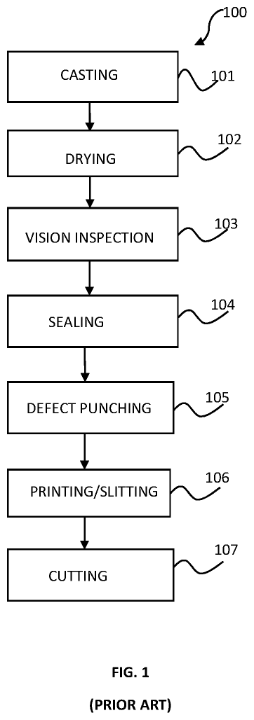

FIG. 2 is a flow chart of a coordinated matrix manufacturing of a pharmaceutical formula in ODF form in accordance with an exemplary embodiment of the present invention;

FIG. 3 is a flow chart of a self-correcting process of the coordinated matrix manufacturing of a pharmaceutical formula in ODF form in accordance with an exemplary embodiment of the present invention; and

FIG. 4A-FIG. 4B illustrate different geometrical shapes of ODF in the coordinated matrix manufacturing in accordance with an exemplary embodiment of the present invention.

The figures depict various embodiments of the technology for the purposes of illustration only. A person of ordinary skill in the art will readily recognize from the following discussion that alternative embodiments of the structures and methods illustrated herein may be employed without departing from the principles of the technology described herein.

### DETAILED DESCRIPTION OF THE INVENTION

Reference will now be made in details to the preferred embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. However, it will be obvious to one of ordinary skill in the art that the present invention may be practiced without these specific details. In other instances, well-known methods, procedures, components, and circuits have not been described in details so as not to unnecessarily obscure aspects of the present invention.

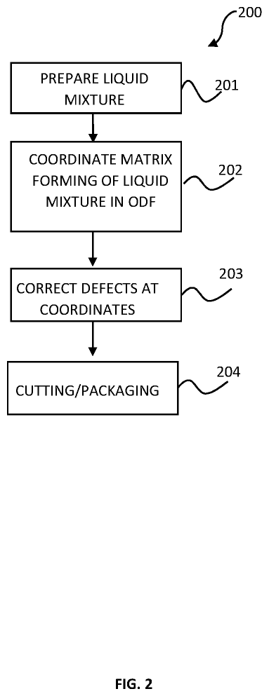

Exemplary embodiments and aspects of the present invention are now described with reference to FIG. 2 to FIG. 4. The present disclosure discloses the following features of the present invention: (1) a method and manufacturing system for producing ODFs of any formula which can detect and correct defects without discarding any of them, (2) a method and manufacturing system for producing ODF of any formulas that involves fewer steps than the conventional casting production method, (3) a method and manufacturing system for producing ODF of any formulas that can conveniently control the geometrical shapes, number, and dimension of each ODF product, and (4) a method and manufacturing system for producing ODFs of any formula that uses high-technology printing methods such as 4D printing and self-configuring materials.

Now referring to FIG. 2, FIG. 2 is a flow chart illustrating a method 200 for manufacturing ODF products in accordance with an exemplary embodiment of the present invention. In a generalized structure of the present invention, food identification method 200 includes 4 major components: a preparation of a product formula step 201, coordinated matrix forming step 202 of the product formula in ODF format, a defect correction step 203, and a packaging step 204.

At preparation step 201 includes mixing the formula product conducive to the ODF format. In various aspects of the present invention, the formula product is a sub-lingual heat resistant and antioxidant cannabinoid mixture which comprises tetrahydrocannabinol (THC) having a first predetermined percentage (%) by weight, a cannabidiol (CBD) having a second predetermined percentage (%) by weight nixed with saturated fatty acids such as stearic adds. More particularly, the mixture includes cannabinoids compounds 203 (C₄₁H₆₅O₂) and 213 (C₆₁H₁₀₆O₂) and coating cellulose that is oil insoluble and water soluble excipient polymer such as hydroxyl propyl methyl cellulose (HPMC), cellulose derivative gelatin, and/or pullulan described in a patent application entitled, “Heat and Oxidation Resistance Tetrahydrocannabinol and Cannabidiol (CBD) Compound and Method of Manufacturing the Same”. The patent applications identified above is incorporated herewith by reference in its entirety to provide continuity of disclosure.

At step 202, the formula composition is coordinated matrix formed in an array of ODF units. Each ODF unit has a matrix address such as column and row address. Each droplet that constitutes the ODF unit has xyz-coordinates. For ex ample, each droplet is similar to a pixel of a picture that has xyz-coordinates. If an ODF unit is dimensioned as 100×200, then there are 20,000 droplets that constitutes the ODF unit, each droplet having its own xyz-coordinates. In various embodiments of the present invention, step 202 is implemented using a 2D dot printer, a 3D printer, and a 4D printer.

At step 203, detected defects are corrected by reforming those ODF units that are defected at the matrix addresses and at the xyz-coordinates of the defected droplets. Step 203 is implemented by a controller operative to keep tracks of the dimension, geometrical shapes, matrix addresses, and droplet xyz-coordinates of each ODF unit. This controller controls the coordinated matrix forming device such as 2D, 3D, and 4D printers.

At step 204, after all ODF units are scanned and defects are cured, ODF units are cut and packaged.

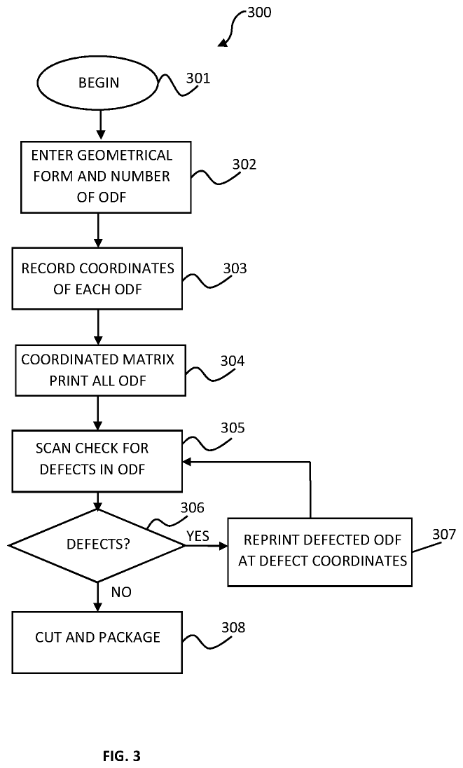

Now referring to FIG. 3, a flow chart of a self-correcting process 300 of the coordinated matrix manufacturing of a pharmaceutical formula in ODF form in accordance with an exemplary embodiment of the present invention is presented. It is noted that self-correcting process 300 is applicable to cannabis medicine, supplementary foods, and other pharmaceutical formulas for both human and animals.

At step 301, the self-correcting process 300 begins. In various implementations of step 301, a mixture such as sub-lingual cannabis product which includes the formula product is a sub-lingual heat resistant and antioxidant cannabinoid mixture which comprises tetrahydrocannabinol (THC) having a first predetermined percentage (%) by weight, a cannabidiol (CBD) having a second predetermined percentage (%) by weight mixed with saturated fatty acids such as olive oils. In other implementations, printable polymer is mixed with the heat resistant and antioxidant cannabinoid mixture is prepared so that ODF can be produced using printer devices. In other implementations, form-configuring materials are mixed with the heat resistant and antioxidant cannabinoid mixture for 4D printer so that the ODF he heat resistant and antioxidant cannabinoid mixture is conformed to the shape of the tongue and dissolve quickly when stimulated by saliva.

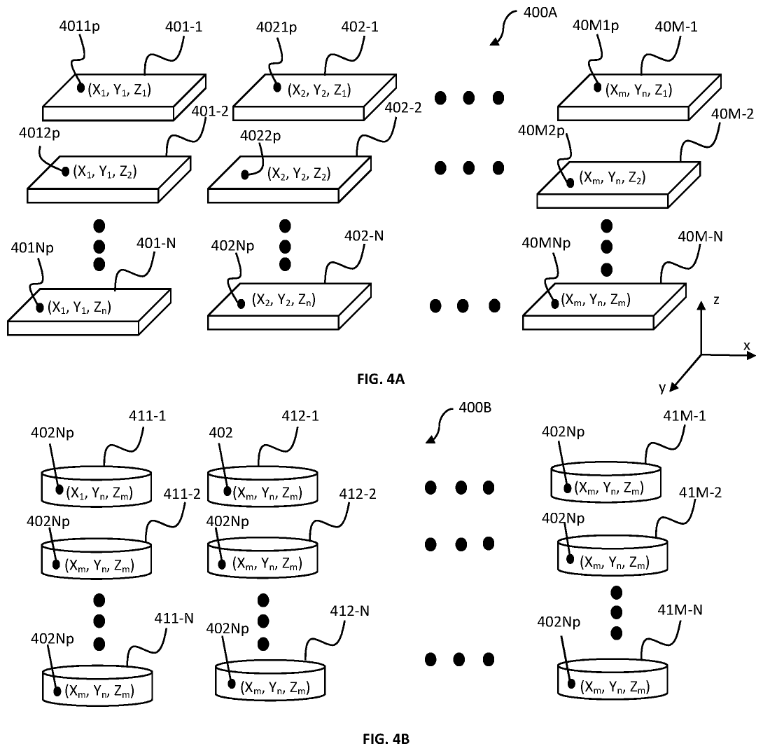

At step 302, the geometrical shape and number of ODF units are entered. In the present invention, the shape and number of OBF units can be determined. For example, instead of a rectangular ODF unit, the implementation of step 302 can produce 3D pill shape, oval shape, cylindrical shape, animal shapes, leaf shapes, or any shapes that the manufacturer prefers. For example, children prefers animal shape OBD units. In addition, the thickness, and dimension, and the array size of ODF units can be precisely controlled. Please refer to FIG. 4 for illustrative implementations of step 302.

At step 303, the matrix having column and row addresses as well as xyz-coordinates of each droplet of each ODF unit are recorded. Step 303 is implemented by a controller. Any high-speed microprocessors from AMD or Intel available in the market can be used to implement step 303.

Next, at step 304, the ODF units in form of coordinated matrix are printed. In some implementations of step 304, a printer including 2D, 3D, or 4D printer is used. 2D printer can be a dot matrix printer. 3D printer adds thickness to the 2D printer. 4D printer enables ODF units to dissolve and change shape upon contact with saliva. As a non-limiting example of steps 303 and 304, if 100 ODF units of the heat resistant and antioxidant cannabinoid mixture are to be manufactured. A 10×10 matrix of ODF units is entered. The geometrical shape of each ODF unit can be selected. If a rectangular shape is chosen, the width, the length, the thickness, and the resolution of each ODF are also entered. As alluded above, a 1×0.5×0.005 cm ODF unit having a droplet resolution of 100×200 are entered. That is, each ODF unit is constituted by 20,000 droplets of CBD material, each having xyz-coordinates.

At step 305, the ODF units are scanned for defects. Defects include, but not limited to, air bubbles, non-uniform thickness, smears, etc. In various implementation of step 305, optical scanners can be used.

At step 306, if no defects are found, process 100 moves to step 308.

At step 307, if defects are found, the exact matrix addresses and xyz-coordinates of defects are recorded. Step 307 reprints any defective ODF units at their matrix addresses and xyz-coordinates where defects are located. Step 307 helps achieving high efficiency because defective ODF units are reprinted without being discarded. Steps 305-307 are repeated until all defects are fixed.

Finally, at step 308, when no defects are found, the ODF units are cut and packaged.

Finally referring to FIG. 4A and FIG. 4B illustrating different geometrical shapes of ODF in the coordinated matrix manufacturing in accordance with an exemplary embodiment of the present invention are presented.

Referring to FIG. 4A, an N×M matrix 400A of ODF units in form of 3D rectangular shape is illustrated. Matrix 400A has N rows and M columns, where M and N are non-zero positive integers. Each ODF unit has a matrix address of row and column. Matrix 400A contains: in the first row, a ODF unit 401-1, 402-1, . . . , and 40M-1; in the second row, a ODF unit 401-2, 402-2, . . . , and 40M-2; and in the Nth row (bottom row), a ODF unit 401-N, 402-N, . . . , and 40M-N. In the first row, ODF unit 401-1 is constituted by droplets 4011p each having xyz coordinates (xi, yi, zi). Similarly, ODF unit 402-1 has droplets 4021p each having xyz coordinates (xi, yi, zi), ODF unit 40M-1 has droplets 40M1p each having xyz coordinates (xi, yi, zi). In the second row, ODF unit 401-2 is constituted by droplets 4012p each having xyz coordinates (xi, yi, zi). Similarly, ODF unit 402-2 has droplets 4022p each having xyz coordinates (xi, yi, zi), ODF unit 40M-2 has droplets 40M2p each having xyz coordinates (xi, yi, zi). In the last row, ODF unit 401-N is constituted by droplets 401Np each having xyz coordinates (xi, yi, zi). Similarly, ODF unit 402-N has droplets 402Np each having xyz coordinates (xi, yi, zi), ODF unit 40M-N has droplets 40MNp each having xyz coordinates (xi, yi, zi). That way, any defective ODF units at any particular matrix location can be reprinted and corrected. It is noted that each unit in matrix 400A described above includes cannabinoid medication 300 as described in a patent application entitled, “Heat And Oxidation Resistant Δ9 Tetrahydrocannobinol (THC) And Cannabinol (CBN) Compound And Method Of Manufacturing the Same”, filed on Dec. 18, 2019. The patent application identified above is incorporated herewith by reference in its entirety to provide continuity of disclosure. Briefly, cannabinoid formula 300 includes a cannabinoid compound 302 coated by an edible-water-dissolvable polymer 301 (“coating layer 301”). In many embodiments of the present invention, cannabinoid compound 302 is either compound 203 (C₄₁H₆₅O₂), compound 213 (C₆₁H₁₀₆O₂). In other embodiments, cannabinoid compound 302 also includes terpenes, CBN reacted with saturated fatty acids as described above in method 100. Coating layer 301 includes hydroxyl propyl methyl cellulose (HPMC), polyglycerol polyricinoleate (PGPR), cellulose derivative gelatin, and/or pullulan. Coating layer 301 is a water dissolvable and bioavailable protective layer that prevent deteriorating agent from degrading cannabinoid compound 302.
Indifferent embodiments, each unit 411-1 . . . 41M-N, also includes peppermint oil and other flavonoids.

Now referring to FIG. 4B, an N×M matrix 400B of ODF units in form of 3D oval shape is illustrated. Matrix 400B has N rows and M columns, where M and N are non-zero positive integers. Each ODF unit has a matrix address of row and column. Matrix 400B contains: in the first row, a ODF unit 411-1, 402-1, . . . , and 41M-1; in the second row, a ODF unit 411-2, 412-2, . . . , and 41M-2; and in the Nth row (bottom row), a ODF unit 411-N, 402-N, . . . , and 41M-N. In the first row, ODF unit 411-1 is constituted by droplets 4111p each having xyz coordinates (xi, yi, zi). Similarly, ODF unit 412-1 has droplets 4121p each having xyz coordinates (xi, yi, zi), ODF unit 41M-1 has droplets 41M1p each having xyz coordinates (xi, yi, zi). In the second row, ODF unit 411-2 is constituted by droplets 4112p each having xyz coordinates (xi, yi, zi). Similarly, ODF unit 412-2 has droplets 4122p each having xyz coordinates (xi, yi, zi), ODF unit 41M-2 has droplets 41M2p each having xyz coordinates (xi, yi, zi). In the last row, ODF unit 411-N is constituted by droplets 411Np each having xyz coordinates (xi, yi, zi). Similarly, ODF unit 412-N has droplets 412Np each having xyz coordinates (xi, yi, zi), ODF unit 41M-N has droplets 41MNp each having xyz coordinates (xi, yi, zi). That way, any defective ODF units at any particular matrix location can be reprinted and corrected. It is noted that each unit in matrix 400B described above includes cannabinoid medication 300 as described in a patent application entitled, “Heat And Oxidation Resistant Δ9 Tetrahydrocannobinol (THC) And Cannabinol (CBN) Compound And Method Of Manufacturing the Same”, filed on Dec. 18, 2019. The patent application identified above is incorporated herewith by reference in its entirety to provide continuity of disclosure. Briefly, cannabinoid formula 300 includes a cannabinoid compound 302 coated by an edible-water-dissolvable polymer 301 (“coating layer 301”). In many embodiments of the present invention, cannabinoid compound 302 is either compound 203 (C₄₁H₆₅O₂), compound 213 (C₆₁H₁₀₆O₂). In other embodiments, cannabinoid compound 302 also includes terpenes, CBN reacted with saturated fatty acids as described above in method 100. Coating layer 301 includes hydroxyl propyl methyl cellulose (HPMC), polyglycerol polyricinoleate (PGPR), cellulose derivative gelatin, and/or pullulan. Coating layer 301 is a water dissolvable and bioavailable protective layer that prevent deteriorating agent from degrading cannabinoid compound 302.

From the forgoing disclosure, method 200 and method 300 of the present invention achieves the following objectives:
a fewer steps and more cost-effective manufacturing process than the prior art casting production to manufacture ODF products.
a system and process of manufacturing ODF products that can correct defects without discarding them, thus improving efficiency.
a system and process of manufacturing ODF products that can control the numbers, the dimension, and the geographical shapes of the ODF products.
a system and process of manufacturing ODF products that can use advanced printing technology such as 4D printing.

The manufacturing processes such as 200 and 300 may be implemented in a non-transitory computer software programs stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The disclosed flowchart and block diagrams illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The flow diagrams depicted herein are just one example. There may be many variations to this diagram or the steps (or operations) described therein without departing from the spirit of the invention. For instance, the steps may be performed in a differing order or steps may be added, deleted or modified. All of these variations are considered a part of the claimed invention.

While the preferred embodiment to the invention had been described, it will be understood that those skilled in the art, both now and in the future, may make various improvements and enhancements which fall within the scope of the claims which follow. These claims should be construed to maintain the proper protection for the invention first described.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

1. A system for producing a formula in form of orally disintegrating film (ODF), comprising:
a heat and oxidation resistant Tetrahydrocannabinol (THC) and Cannabidiol composition further comprising: a cannabidiol element (CBD) having a first predetermine percentage (%) by weight;
a Δ⁹ tetrahydrocannabidiol (THC) element having a second predetermine percentage (%) by weight (w/w); and
fatty acids having a third predetermined percentage wherein said fatty acids are consisted of single carbon bond with a formula of CH₃(CH₂)ₙCOOH;
a printer for generating said formula in form of a matrix ODF, wherein said matrix ODF further comprises a plurality of ODF units each containing said formula and having xyz-coordinates; and
a scanner for detecting defects in said plurality of ODF units.

2. (canceled)

3. The system of claim 1 further comprising a controller operative to:
record said xyz-coordinates for said ODF units where said defects occur;
return ODF units that contain defects to said printer for fixing said defects; and
instruct said printer to reprint said ODF units at said xyz-coordinates where said defects are detected.

4. The system of claim 3 wherein said controller is operative to receive a geometrical shape and number of said plurality of ODF units in order to assign said xyz-coordinates to each of said plurality of ODF units.

5. The system of claim 3 wherein printer further comprises a 2 dimension (2D) dot matrix printer.

6. The system of claim 5 wherein said 2D dot matrix printer prints out said plurality of ODF units by repeatedly depositing a dot of said formula on a substrate until said plurality of ODF units is complete, wherein said dot of said formula has said xyz-coordinates.

7. The system of claim 3 wherein said printer further comprises a 3 dimension (3D) printer.

8. The system of claim 7 wherein said 3D printer prints out said plurality of ODF units upon receiving a width, a length, and a thickness of each of said ODF unit from said controller.

9. The system of claim 3 wherein said printer further comprises a 4 dimension (4D) printer.

10. The system of claim 9 wherein said 4D printer further receiving a self-reconfiguring material added to dissolve said plurality of ODF units upon contact with saliva.
